(19) United States
(12) Patent Application Publication
(10) Pub. No.: US 2012/0245565 A1
(43) Pub. Date: Sep. 27, 2012

(54) METHOD FOR DELIVERING GENE AND CELL THERAPY TO A TUMOR OR TARGETED SITE USING AN IMPLANTED METRONOMIC BIOFEEDBACK PUMP

(75) Inventors: Yehoshua Shachar, Santa Monica, CA (US); Thomas C. Chen, La Canada, CA (US); Winston Wu, Alhambra, CA (US); Brett Jordan, Los Angeles, CA (US); Herwin Chan, Torrance, CA (US); Kyle Zimmerman, Los Angeles, CA (US); Paladin Luboff, Los Angeles, CA (US)

(73) Assignee: PHARMACO-KINESIS CORPORATION, Inglewood, CA (US)

(21) Appl. No.: 13/052,811

(22) Filed: Mar. 21, 2011

(51) Int. Cl. A61M 37/00

(52) U.S. Cl. 604/891.1

(57) ABSTRACT

A method for utilizing a controlled pump implanted into a patient connected to a multilumen catheter allowing delivery to and sampling from the brain or other organ for treatment of a cancer. The pump delivers a plurality of medicating agents, including viral and non-viral vectors for gene therapy and cell therapy at a controlled rate, corresponding to the specific needs of the patient. A catheter is implanted in or adjacent to the tumoral region. Fluid drawn from the tumor region to the pump via the multilumen catheter is analyzed within the pump by various biofeedback sensors. The operation of the apparatus and hence the treatment is remotely controlled based on these measurements and displayed through an external controller. The method allows localized delivery of gene and cell therapy to a solid tumor or tumoral region, or to any other treatment area of interest within the patient.

### RELATED APPLICATIONS

This application is related to the material presented in U.S. Pat. No. 7,799,012, titled ‘A Magnetic Breather Pump and a Method for Treating a Brain Tumor Using the Same’, issued Sep. 21, 2010.

### FIELD OF THE INVENTION

The invention relates to the field of implantable therapeutic agent delivery systems, specifically an implanted metronomic pump, and a method for delivering gene and cell therapy into a tumor or targeted organ using the same.

### BACKGROUND

Cancer is an often pernicious disease for which there is no absolute cure. However, there are several options for therapeutic treatments available when tumors develop inside the human body. Currently approved methods of treatment include surgical removal of the tumor, radiation therapy, and chemotherapy. In addition, various investigators are developing and performing clinical trials on gene therapy (utilizing viral and non-viral vectors) and cell therapy techniques. Each of these options for treatment will be discussed further below.

The first option for treatment is to surgically remove the tumor. This is the oldest and most direct way for treating a tumor. Surgery can cure some varieties of cancer if performed before the cancer metastasizes. Surgery is effective in obtaining tissue diagnosis and removing the mass effect of the tumor from the adjacent normal tissue. For example, in neurological cancer, the mass effect of tumoral growth becomes increasingly important. The cranial cavity and spinal column do not have enough available space to accommodate a large tumoral mass. As the tumor grows, it compresses healthy nerve or brain tissue. Unrestricted tumoral growth will cause pain to the patient, and can eventually cause disability or death.

However, surgery is invasive, expensive, and poses potential complications for the patient. Most importantly, surgery cannot cure certain types of cancer. This is especially true with a malignant brain tumor, as the cancer cells have often invaded far into the normal brain when the diagnosis is first confirmed. Thus it is impossible to guarantee that surgical removal of a brain tumor has eliminated all malignant cellular tissue. Additionally, surgery is only available when the tumor is in a surgically accessible location. For example, tumors located deep within the brain are often inoperable as the surgery would significantly impair the patient's neurological function. Even if surgery is possible, there is still a chance of irreparable tissue damage and an extremely long recovery time associated with surgery. Additionally, any organs (or parts of organs) that are excised during the surgery diminish the normal functioning of the patient.

The second treatment option is to utilize radiation therapy. This therapy can be either localized (i.e. stereotactic) or global (i.e. total body irradiation) in nature. Radiation therapy utilizes ionizing radiation to control malignant cells by damaging the genetic material of cellular tissue. Because cancer cells replicate quickly, they are damaged to a greater extent than normal cellular tissue. For brain cancer, radiation therapy is usually given as a fractionated dosage treatment, covering a certain field encompassing the tumor, over a period of six weeks. Spatially localized forms of radiation, including cyberknife and gamma knife have been used with varying levels of success.

However, greater adverse reactions in the patient come with more global or higher dose radiation therapy treatments. The most common side effects of radiation therapy include skin rash, permanent skin damage, and fatigue, which can be minimized with localized treatment. While radiation is still widely acknowledged as the most effective mode of adjunctive treatment for a malignant brain tumor, it suffers from the disadvantage of limited fractions and applications, as the brain can only be radiated so much without developing severe sequelae.

The third treatment option is administration of chemotherapy. Chemotherapy is treatment of the tumor by chemicals or drugs to create a cytotoxic standardized treatment regime. There are a great variety of chemotherapeutic agents. Some of the categories of chemotherapeutic agents include; alkylating agents, antimetabolites, anthracyclines, plant alkaloids, and topoisomerase inhibitors. All of these chemicals function by affecting cell division or DNA synthesis.

Because chemotherapeutic agents target cell division or DNA synthesis, they do not affect cancerous cells with great specificity. Cancer cells may develop drug resistance to the chemotherapy, and the use of large doses of toxic agents often leads to serious and debilitating side effects. Patients may experience sequela so severe that use of chemotherapy is no longer a viable treatment option. Higher doses of chemotherapy delivered to tissues creates a more effective treatment regime, while also making the adverse effects to the patient more prominent.

Chemotherapy can be delivered to the patient locally or systemically. Common delivery methods include systemic delivery via intravenous lines, direct intratumoral injections, shunts (e.g. intracranial shunts used to deliver chemotherapies to brain cancers), catheters, and surgically implantable wafers. Chemotherapy is often used as an adjunct to radiation and surgery.

Systemic delivery of chemotherapy is effective in treating malignancy, requires large doses which cause significant adverse effects to the patient. Also, the patient's natural barriers, such as the mesothelium, extracellular matrix (ECM), and blood brain barrier, block transmission of chemotherapy to some organs if delivered systemically.

Localized delivery of chemotherapy can be accomplished utilizing stereotactic injections or chemotherapy wafers (Gliadel, also known as Carmustine or BCNU). Both of these options offer localized delivery, but very little diffusion capability into the tumor or tumor bed. Also, to re-administer medications, the patient will require additional surgical procedures, with their attendant risks and discomfort. Alternatively, chemotherapy can be delivered to an organ through an externalized pump and a catheter or shunt. While this is an effective way to administer medication and can utilize convection enhanced delivery, the procedure also carries a serious risk of life-threatening infection. This is because the administration equipment maintains an open wound through which to deliver the drug during the procedure, which is usually delivered for a cycle of 4 to 6 days.

Gene therapy offers an alternative modality to treatment of cancer. Gene therapy treatments for cancer lack FDA approval at this Lime, but many are in their clinical trial phases and show great promise. Numerous companies have invested significant research into various gene therapies. Several of these companies are: Onyx Pharmaceuticals, Inc.; Tocagen, Inc.; and Ark Therapeutics (originally Eurogene Ltd.). The general principles of gene therapy will be summarized below, followed by specific cancer gene therapy examples.

Generally, gene therapy involves the insertion of genetic material into an individual patient's tissues and cells to treat disease. The medium of transmission of the genetic material into the cell is called a vector. Vectors are gene delivery vehicles utilized to deliver DNA or RNA into host cells. Once inside of the cellular membrane, each vector will utilize one specific mechanism to regulate gene expression. Some vectors (e.g. retrovirus vectors) allow for genetic material to be inserted into the DNA of the host cell (thereby changing the genetic code of that cell and its progeny). Alternatively, some vectors (e.g. adenoviruses) do not incorporate their DNA into the host cell's genetic code. Adenoviruses float freely within the cell nucleus, where they are transcribed just like any other gene, but do not become part of the genetic code of the host cell, and are not passed on to daughter cells.

Gene therapy is capable of targeting any DNA or RNA expression, including nuclear DNA (i.e. chromatin) and mitochondrial DNA. The category of gene therapy vectors includes the categories of viral and non-viral vectors. Within each category, there are various sub-categories of vector types, each with its own unique properties. Differentiating between more naturally occurring vectors (i.e. the retrovirus HIV) and purely synthesized vectors (i.e. dendrimers) is not of great importance. The trend in gene therapy is to utilize known vectors as a template, and modify them as needed. The vectors utilized in gene therapy currently are summarized below.

Viral vectors include: (a) retroviruses, which includes the subclass lentivirus, viruses that contain RNA and utilize reverse transcriptase to insert their genetic material into the host cell genome; (b) adenoviruses, which are a non-developed (naked), double-stranded, linear DNA family, which do not integrate into the genome and are not replicated during cell division; (c) adeno-associated viruses, single-stranded DNA viruses that can infect both dividing and non-dividing cells (nerve cells do not replicate in adults), and can incorporate their genome into the host cell; (d) herpes simplex viruses which take advantage of the neural tropism of the virus (e) man-made viruses, which are usually hybrid viruses formed from a combination of the prior virus classes utilizing envelope-protein-pseudotyping of the viral vectors (i.e. VSV G-pseudotyped lentivirus) to foster greater cell targeting accuracy.

An important consideration in use of viral vector is whether the vector is replication-competent or replication-incompetent. Replication-competent viral vectors can reproduce themselves within the patient's body by utilizing the same mechanism as a naturally occurring virus. This mechanism minimizes the necessity for repeated gene therapy treatment. If designed and targeted correctly, one dose of the virus could replicate through the patient's body until every targeted cell has received therapy. On the other hand, replication incompetent viral vector does not reproduce within the patient's body. Each viral vector has a chance of transfecting a maximum of one cell in the patient. If a wider or more effective dose is needed, the gene therapy treatment must be repeated. However, replication-incompetent virus vectors are deemed safer, because of their lack of ability to replicate, which allows for a more controlled effect.

Non-viral vectors include: (a) naked DNA, which is nothing more than simple DNA molecules, which lacks a protective coating, and which is often used in conjunction other cell insertion techniques (i.e. electroporation, sonoporation, or a “gene gun” with DNA-coated gold particles delivered by high pressure gas) in order to assist in its otherwise low transmission rate; (b) Oligonucleotides, which are short nucleic acid polymer chains (RNA or DNA) that are often synthesized for use as antisense treatments to target specific nucleic sequences and thereby diminish their expression; (c) lipoplexes, which are synthesized liposome envelopes that are complexed with DNA, and can be either anionic, neutral, or cationic in nature; (d) polyplexes, which are usually cationic polymers complexed with DNA, and differ primarily from lipoplexes in that polyplexes often require co-transfection with endosome-lytic agents such as an adenovirus; (e) dendrimers (e.g. Dendritic Nanotechnologies in Michigan discovered Priostar dendrimers), synthesized macromolecules capable of having a water-soluble (cationic) molecule with internal hydrophobicity (anionic), allowing encapsulation of hydrophobic drugs into a cell via endocytosis with a large degree of targeting specificity; (e) hybrid methods for creation of vectors, for example, virosomes, which combine liposomes with an inactivated HIV or influenza virus and (f) nanoengineered substances such as Ormosil (organically modified silicate), which utilizes silica and has a high transfection efficiency rate. (See: S. Li at al., Nonviral gene therapy: promises and challenges, Gene Therapy, Vol. 7, pp 31-34, 2000.) Bacteria can also be used as a transduction vector for genetic material. (R. Palffy at al., Bacteria in gene therapy: bactofection versus alternative gene therapy, Gene Therapy, Vol. 13, pp. 101-105, 2006.)

Gene therapy targeting can be accomplished either through a cell-specific targeted vector delivered systemically, or through local delivery of a vector. All gene therapy vectors have some level of cell transduction specificity, some much more so than others.

Systemic delivery is usually accomplished via an intravenous line or injection into the blood stream. This procedure is generally performed in a hospital. A relatively large amount of vector must be given systemically in order for a small amount to arrive directly at the tumor site. This is even more the case for brain tumors, because of the necessity in crossing a partially broken down blood brain barrier. In the case of brain cancer, intravenous systemic delivery is often limited to the luminal side of the blood vessels within the brain. This hampers delivery of gene therapy to the tumoral mass itself.

Because of the inability to effectively deliver gene therapy throughout the tumor, current gene therapy treatments utilize sophisticated chemical and biological targeting mechanisms. For example, many oncolytic viruses employ protein specificity to target malignant cellular tissue. Alternatively, other therapy modalities utilize matrix metalloproteinase to assist vectors in crossing the extracellular matrix into the patient's cells. (Mikala Egeblad et al., New functions for the matrix metalloproteinases in cancer progression, Cancer, Vol. 2., pp. 151-174, March 2002. See also: Hideaki Nagase et al., Matrix Metalloproteinases, The Journal of Biological Chemistry, Vol. 274, No. 31, pp. 21491-21494, July 1999.) These techniques assist in overcoming the patient's natural protective barriers that affect delivery of therapies to non-neural cancers, for example, the mesothelial membrane lining body cavities, the blood brain barrier; and the extracellular matrices within the body. (James P. Basilion Ph.D., et al., Gene therapy of brain tumors: problems presented by physiological barriers, Neurosurg. Focus, Vol, 8., No. 4, Article 2, pp. 1-7, April 2000.) Further, larger doses of gene therapy vector are highly expensive, and can cause a greater immune response. Also, the larger the number of vectors the patient is exposed to, the higher the chances of significant side effects, such as tumor growth (benign or malignant) due to the insertional mutagenesis. However, these sophisticated targeting and transport mechanisms target non-cancerous cells as well, especially when gene therapy is administered systemically to the patient.

Alternatively, gene therapy can be delivered locally. Under several of the therapies in clinical trial, this is accomplished with a stereotactic injection. The injection is usually administered directly into the tumor, or into the resection cavity directly after surgery. This can be effective in minimizing adverse reactions and minimizing required effective dosage. However, stereotactic injection is limited because it must be administered in a hospital by a specialized clinician, and carries the attendant risks of a minor surgical procedure. This is especially the case in regards to brain cancers such as malignant glioma, where a first dose may be delivered after a standard surgical rescission excision of the tumor, but a second stereotactic administration of gene therapy agent would require an additional surgical procedure. In one study, completed in 2000, gene therapy was delivered to patients with glioma with no significant side-effects, but with no significant increase in the survival of the patients. In the paper describing this phase III clinical study, the author theorizes that a reason that there was no significant benefit from the treatment was because of a lack of proper delivery system. (“A Phase Clinical Evaluation of Herpes Simplex Virus Type 1 Thymidine Kinase and Ganciclovir Gene Therapy as an Adjuvant to Surgical Resection and Radiation in Adults with Previously Untreated Glioblastoma Multiforme”, by Rainov, N. G., HUMAN GENE THERAPY 11:2389-2401 Nov. 20, 2000.) Moreover, the spread of the gene therapy solution is limited to the injection site and only a few millimeters of the adjacent brain matter.

Cell therapy is the process of introducing whole cells into a tissue in order to provide treatment for a malady or disease. Currently, cell therapy will commonly use gene therapy techniques in order to increase biocompatibility and translatability before the cells are introduced into the patient's body. The problems with delivery of gene therapy throughout the tumor also apply to cell therapy. Cell therapy uses several sources of implantable material, including: stem cells (allogeneic or autologous), including mesenchymal stem cells; animal sources for xenotransplantation; the patient's own differentiated cells utilized to create transdifferentiated cells; and modified human cells (allogeneic). One currently utilized technique can be bone marrow transplant for cancer patients with severely compromised immune systems. A patient's bone marrow may be compromised by cancer treatments, especially when radiation therapy and chemotherapy are utilized. Additionally, a promising cell therapy technique in its clinical phase is the use of mesenchymal stem cell transplantation in order to re-enervate the denervated striatum of the brain of patients with Parkinson's disease. (D. Baksh et al., Adult mesenchymal stein cells: characterization, differentiation, and application in cell and gene therapy, J. Cell. Mol. Med., Vol. 8, No. 3, pp. 301-316, 2004.) This therapy has already undergone proof-of-principle testing, and allows for the possibility of organ restoration to allow normal function for a patient after otherwise debilitating cancer treatments or significant tumoral tissue growth.

Cell therapies are currently generally delivered locally via stereotactic injection, with its advantages and drawbacks being the same as discussed above under the subjects of chemotherapy and gene therapy treatments. However, none of the cell therapy techniques currently in practice include a method of metronomic delivery of cell therapy to a localized treatment site without subjecting the patient to repeated surgeries and injections. Many cell therapy techniques utilize the natural tropism or migration of its carrying vector. For instance, neural stem cells are utilized as carrying agents for cytotoxic drugs or genes because of their tropism for migration towards a brain tumor. Cell therapy treatments could be significantly improved through a delivery system that allows for metronomic localized delivery of treatment utilizing convection enhanced delivery to spread the treatment farther and with more precision. (Krys S. Bankiewicz et al., Convection-Enhanced Delivery of AAV Vector in Parkinsonian Monkeys; In Vivo Detection of Gene Expression and Restoration of Dopaminergic Function Using Pro-drug Approach, Experimental Neurology, Vol. 164, pp 2-14, 2000.)

There are a great plurality of tests that can be utilized to determine the state of a cancer in the patient. Tumor markers can be tested in lab work to help determine the state of a tumor. (Bigbee W, Herberman R B. Tumor markers and immunodiagnosis. In: Kufe D W, Pollock R E, Weichselbaum R R, Bast R C, Gansler T S, Holland J F, Frei E eds. Cancer Medicine. 6th ed. Hamilton, Ontario: B C Decker; 2003: 209-220.) Other factors, such as vascular endothelial growth factor (VEGF) can be utilized and tested to determine the state of the cancer. However, there is nothing that incorporates the ability to do such analysis at a tumor site, combined with a pumping device that will deliver treatment.

Currently, systemic immunosuppressive therapy is not used in conjunction with gene therapy studies. Several of the gene therapies currently in clinical development utilize another therapeutic agent as adjunctive treatment, including chemotherapy and radiation for treatment of glioblastoma.

As demonstrated above, both gene and cell therapy suffer from the problem of adequate delivery, whether by systemic or local delivery. Modern molecular biology has allowed for the creation of more and more sophisticated viruses and cell therapy. As a result, when a gene or cell therapy trial fails, the question always remains whether the therapy did not work because of the product being delivered, or because of the poor delivery system. Recently, there have been developments in the field of medical drug delivery systems that may help to resolve this issue. The majority of these systems have taken the form of a pump. These devices release a variety of drugs into various positions in the body of a patient. Here, we propose the use of the metronomic biofeedback pump, an implantable intratumoral pump which can be used to metronomically deliver both gene and cell therapy under positive pressure into the tumor and tumor bed. This process enables rapid delivery of the virus throughout the tumor, bypassing the need for the virus to digest its way through the extracellular matrix. Moreover, even in the case of replication competent viruses, viral spread by replication is often not possible secondary to the tremendous heterogeneity of the tumor microenvironment (including necrotic and hypoxic cells which are not actively dividing). Therefore, a novel method allowing viral spread throughout the tumor utilizing an internalized pump to deliver it will be crucial to further advancement and success of the field.

A summary of prior art of delivery pumps follows. U.S. Pat. Nos. 6,852,104 (“Blonquist”) and 6,659,978 (“Kasuga”) comprise a small tank for holding a drug regimen, a pump for pumping the drug regimen into the body of a patient, and some sort of electronic control system that allows the user to program the specific amount and time a certain drug regiment is to be administered. These apparatus may be ideal for administering certain drugs, such as insulin and pain medication. However, they are neither designed nor suitable for directly treating a tumor within a patient.

Other prior art examples such as U.S. Pat. Nos. 5,242,406 (“Gross”) and 6,571,125 (“Thompson”) offer smaller, more convenient alternatives for administering drugs, however their reliance on maintaining a specific set of pressures and a certain amount of electrical current respectively makes them too complicated and prone to error.

U.S. Pat. No. 6,893,429 (Peterson) disclose a pump capable of convection enhanced delivery of chemotherapy via multiple catheters to the brain. However, this prior art does not utilize multiple ampoules for multiple therapeutic options, a catheter for sampling the treatment site fluids, a lab-on-a-chip for analysis internal to the pumping mechanism, RF communication with the pumping device allowing adjustable treatment regimes, or the MBP mechanism and its plethora of needles to enhance delivery area.

U.S. Pat. No. 3,721,681 (“Blackshear”) discloses a pumping device that will distribute at a slow rate, but has no ability to adjust the treatment throughout the course of therapy to maximize the effectiveness to the patient. Further, the pump does not allow a feedback loop utilizing analysis from the tumor site.

U.S. Pat. Nos. 5,702,384 (“Umeyama”) and 5,501,662 (“Hofmann”) discloses a device utilized for distributing gene therapy or pharmacological compounds systemically into the blood stream. However, this device does not provide for localized metronomic delivery of gene or cell therapy intratumorally.

U.S. Pat. Nos. 7,351,239 (“Gill”), 7,288,085 (“Olsen”), and 6,726,678 (“Nelson”) disclose a pump or reservoir that is capable of delivering medicating fluids to the brain, but requires that the pump and drug reservoir be implanted in different locations within the patient. This configuration is not only uncomfortable for the patient, but also increases the possibility of infection and unnecessarily complicates the implanting procedure. Additionally, only one reservoir is taught with these devices, precluding localized combined modality treatment regimes. Finally, none of these devices teach use of cell therapy either individually, or as part of an adjunctive treatment.

What is needed is a device and method for gene and cell therapy that allows for localized metronomic delivery that can be adjusted based upon that specific patient's needs as determined by non-invasive, site-specific testing.

The amalgam of gene therapy and cellular therapy with the current invention allows for a unique combination of drug and device, enabling new treatment options for solid tumor cancers presented in the human body.

### SUMMARY

An implanted pump, named herein as the metronomic biofeedback pump (MBP), capable of metronomically delivering gene therapy or cell therapy with direct feedback on rate of delivery, is implanted with an attached catheter allowing delivery to the brain, organ, or cavity of a patient and delivers a dose of gene therapy or cell therapy solution at a controlled rate corresponding to the specific needs of the patient. The current method is comprised of using a pump containing several bellows, which when contracted, allows gene therapy or cell therapy solution to be pushed out of the bellows into the tubing. When the bellow is contracting, surrounding fluid is pumped out in small quantities (up to 300 μl/minute). Fluid drawn from the patient's tumor can be analyzed, or can be analyzed within the pumping device unit by way of a lab-on-a-chip. The operation of the apparatus, and hence the treatment, is remotely controlled based on these measurements. These measurements are recorded and displayed on an external controller for the clinician.

The illustrated embodiment of the invention used in this method solves the above limitations in the prior art, as well as other problems. This method effectively provides treatment for solid tumors (including brain tumors) utilizing a multi-delivery catheter implanted into a tumor or tumor cavity. An unresectable tumor is a tumor in which a surgical removal of all or part of an organ, tissue, or structure is not practically feasible. An externally controlled, internally implanted pump can deliver multiple therapeutic agents (including gene therapy and cell therapy treatments) at a controlled rate corresponding to the specific needs of the patient.

The current method is for delivering a viral or non-viral gene vector for gene therapy or a therapeutic agent for cell therapy of a tumor in a patient by first surgically implanting a fluid-exchange catheter into a treatment site. A fluid-exchange catheter is then connected to an analyzer-pump unit which is then operated to infuse the viral or nonviral vector or the therapeutic agent stored in a reservoir into the treatment site. A sample of fluid from the treatment site is suctioned out and then transferred to the analyzer-pump unit which monitors the progress of treatment. The treatment can be changed by controlling the analyzer-pump unit and a reservoir containing the viral or nonviral vector or therapeutic agent may be refilled or replaced to provide ongoing treatment.

Operating the analyzer-pump unit includes contracting and then expanding an inner membrane reservoir in the analyzer-pump unit by oscillation of a magnetic solenoid coupled to the inner membrane reservoir to deliver the gene vector or therapeutic agent.

Monitoring the progress of treatment by means of the analyzer-pump unit includes measuring the effectiveness of intratumoral administration and displaying results obtained from the analyzer-pump unit on a display including displaying information related to a determination of effectiveness, an amount of gene vector or therapeutic agent dispensed as a function of time and any flow rate at which the gene vector or therapeutic agent was dispensed.

In another embodiment, the method also includes providing a preoperative simulation using a diffusion model or a convection enhanced delivery model of infusion of the viral or nonviral vector or the therapeutic agent, individually or in combination, to maximize efficiency and minimize toxicity.

In another embodiment, the method step of changing the treatment by controlling the analyzer-pump unit further includes entering a command or data into the analyzer-pump unit from a remote keypad and displaying the commands on a display or sending a command or data to the analyzer-pump unit by means of an RF transceiver and antenna.

In yet another embodiment, the method step of refilling or replacing the reservoir further includes refilling or replacing at least four drug ampules included in the analyzer-pump unit, wherein at least one of the four drug ampules is for gene therapy or cell therapy only.

In an alternative embodiment, the analyzer-pump unit may be cleaned by refilling the reservoir with an ampule of saline solution or a cleansing agent, pumping the saline solution or cleansing agent through the fluidicly communicated portions of the analyzer-pump unit, thereby preparing the analyzer-pump unit to deliver to the patient a substance that would otherwise be incompatible with substances previously administered by the analyzer-pump unit. The contents of the ampules are utilized for combined modality treatments by repetitively cleaning the analyzer-pump unit and refilling or replacing of the reservoir as many times as necessary to utilize a plurality of therapeutic agents including at least one viral or non-viral gene vector for gene therapy or therapeutic agent for cell therapy.

In particular embodiment, the viral vector includes a retrovirus, which includes a virus from the subclass lentivirus; adenovirus; adeno-associated virus; and man-made virus, including a chimera or hybrid virus including a VSV G-pseudotyped lentivirus. The vector may also include either a replication competent or replication incompetent vector. The vector is then utilized in conjunction with a cell insertion technique, including electroporation, sonoporation, or use of a gene gun.

In an alternative embodiment, the non-viral vector comprises naked DNA, an oligonucleotide, a lipoplex, or a polyplex used in conjunction with an endosome-lytic agent, dendrimer, a hybrid method for creation of a vector including a virosome, a nanoengineered substance including an ormosil or a bacteria.

In one particular embodiment, the cell therapy includes the use of an allogeneic or autologous stem cell, a mesenchymal stern cell, an animal source for xenotransplantation or the patient's own differentiated cells utilized to create a transdifferentiated cell.

The invention also provides for an apparatus for delivering a viral or non-viral gene vector for gene therapy or a therapeutic agent for cell therapy of a tumor in a patient including a fluid-exchange catheter adapted for surgical implantation into a treatment site, an analyzer-pump unit fluidicly communicated with the fluid-exchange catheter, and a refillable or replaceable reservoir fluidicly communicated with the analyzer-pump unit to infuse the viral or nonviral vector or the therapeutic agent stored in a reservoir into the treatment site. The analyzer-pump unit suctions a sample of fluid from the treatment site, analyzes the sample to monitor the treatment and is controllable to change the treatment in response to monitoring. The analyzer-pump unit also includes an inner membrane reservoir operable by oscillation of a magnetic solenoid coupled to the inner membrane reservoir to deliver the gene vector or therapeutic agent.

The microdelivery pump system has two main components: a multidelivery catheter implanted in the tumor or delivery site and an analyzer-pump unit, called the metronomic biofeedback pump (MBP), connected to the catheter. The entire unit is self-contained and entirely internalized.

The medication intake line and the cerebrospinal fluid return line are housed within a catheter. The catheter runs underneath the scalp of the patient, and around the back of the head. The catheter is coupled to the analyzer-pump unit.

The analyzer-pump unit is a housing means for several key components of the apparatus. Cerebrospinal and/or tumoral fluid that has returned from the patient passes through a lab-on-a-chip which measures and monitors the vascular endothelial growth factor (VEGF) levels for indications of progress or regression of the patient's tumor burden. Other tumor markers, peptide markers, protein markers, and products of over/under expression of genes can also be monitored by specific lab-on-a-chip functions. Over expression of genes delivered by gene therapy or cell therapy may also be detected. The user or physician operating the apparatus can then adjust or change the treatment regimen the patient is receiving based on these measurements. Also included in the analyzer-pump unit are piezoelectric pumps to send medicating agents, one of which being the gene therapy or cell therapy solution, through the catheter to a selected treatment site in the patient. An RF communication protocol also allows the unit to be controlled by a physician from a remote location. Flash memory chips and an artificial intelligence processor complete the circuitry needed in order to provide the patient with an effective, easy to use apparatus that delivers medicating agents at a set and controlled rate. Finally, the pump includes a long lasting lithium ion battery that powers the unit itself.

Accordingly the present invention may have one or more of the following advantages described by the objects below.

It is therefore an object of the method to provide a patient with constant delivery of the gene or cellular therapy without re-implanting a catheter every time a patient needs to be treated.

It is another object of the method to provide a metronomic continuous delivery of a therapeutic agent.

It is a further object of the method to provide users and physicians in charge of a patient's treatment instant monitoring and feedback of various tumor parameters in order for the patient's treatment to be changed or adjusted accordingly.

It is a further object of the method to provide patients with tumors an effective way of treating their affliction while minimizing the side effects of therapy, including the side-effects associated with the use of gene therapy or cell therapy treatment regimes.

Another object of the method is to regulate the rate of dispensation of the gene or cell therapy solution by modifying the duty cycle of the valves located in the apparatus.

Another object of the method is to provide a treatment specific to the patient by controlling the processes and mechanisms of the pump apparatus. Different treatment regimes would be specified based upon the size, type, location, and condition of the tumor or disease being treated. For instance, a deeply located smaller tumor would benefit from a limited number of metronomic cycles of gene therapy delivery via a catheter.

Another object of the method is to provide scheduling of medicating agents, such as cell therapy, cytotoxic chemotherapy, biological response modifiers, and gene therapy agents based on their toxicity. Treatments will be designed to measurement and adjusted based on such factors as bioavailability, solubility, concentration, and local circulation. All of these measures will improve the approach to the elimination of solid tumors.

Another object of the method is to address the individual differences of various tumors based on the disease stage, immune factors, body weight, age and chronobiology. The apparatus utilized in this method has the ability to modify the local administration of agents, alter dosing amounts, and alter scheduling of doses in real-time based upon biofeedback from the patient.

Another object of the method is to provide an effective mode of administrating a variety of therapeutic substances, either alone or in sequence, for maximal localized effect. For instance, combination therapy utilizing gene therapy and cell therapy might be administered to treat the cancer and then repair the damage caused by tumor growth. Alternatively, other combination therapies would be available utilizing gene and cell therapy with interferons (IFNs), Interleukin-2 (IL-2), monoclonal antibodies, and tumor necrosis factors (TNFs). A programmable and metronomic regimen would utilize the combination of these therapies to maximize treatment success.

Another object of the method is the use of the pumping device as a tool to enhance research and development of new therapeutic substances, such as gene and cell therapy, in animal studies and human clinical studies. This is accomplished by providing feedback on the use, dose, cycle, circadian time effects, and the entire pharmacokinetic and pharmacodynamic behavior of the medicating agents by way of the pump's sensors. This feedback is measured as an objective biological measure of tumor responses to the agents delivered to the patient, and not as verbal reports of symptomology chronicled by the patient. This method will allow for informative biological feedback from animal studies, and will improve upon current methods to allow pharmaceutical companies in designing therapies safely and rapidly.

Another object of this invention is to provide a method and apparatus for local administration of biological response modifiers, cytotoxic chemotherapeutic agents, cell therapy, and gene therapy agents. In combination, these therapies can be used to enhance mechanisms that support in reducing tumor burden and eliminating tumors. Administration of gene therapy via one bellows and chemotherapeutic drugs in another bellows allows for combining gene therapy and chemotherapy all in one treatment. Local administration of therapeutic agents will be used to induce an improved response by the use of biomodulators, which augment the patient's anti-tumor response via production of cytokines. Local administration of therapies is also critical in maximizing effectiveness through: (a) decreasing suppressor mechanisms; (b) increasing the patient's immunological response; (c) limiting the toxicity of such agents by the locality and dosage; (d) maximizing the localized dose to the desired cellular tissues; (e) increasing susceptibility of cells membrane characteristics for improved therapy results at the site; (f) and decreasing the tumor's ability to metastasize.

The above characteristics are measurable elements since dosing and scheduling improves the effectiveness of therapy on malignant cells while it reduces the exposure of toxins and foreign vector agents to normal tissues. For example, one embodiment provides improved immuno-modulation with relatively little immunosuppression from the patient by minimizing and localizing the dose of cell therapy and gene therapy.

Another object of the method is to provide for defining an improved dose and schedule of biological agents to maximize the anti-tumor effects of each agent while not increasing adverse effects in the patient. Treatment modality by the use of combination therapy and local administration of such agents on a specific schedule is one of the benefits of the method.

It is another object of the method to provide operating physicians a technique of treating brain tumors which bypasses the blood brain barrier in distribution of therapies to the tumor site. This can be accomplished through direct injection and delivery of gene and cellular therapy into the glioma tumor, tumor bed, tumor region, or chosen treatment area utilizing localized catheter delivery.

It is another object of the method to provide the operating physicians a technique of treating the tumor site by transplantation of patient-compatible cells, and therefore using cell therapy techniques to repair the damage caused by the tumor growth and restore normal function to the patient. This technique allows for treatment of the tumor cavity and the tumor penumbra of normal cells that are injured by the tumor, the surgical resection, or adjunctive therapy such as radiation or chemotherapy.

It is another object of the method to provide operating physicians a technique of treating organ tumors without the therapies being diluted or hindered by the mesothelium or extracellular matrix (ECM), inhibiting effective viral or cell therapy transport within the ECM. This is accomplished by direct delivery of therapeutic agents into an affected organ or tumor. This object allows for mechanical dispersal of the agent rapidly throughout the tumor, bypassing the extracellular matrix and the heterogeneous tumor microenvironment.

It is another object of the method to provide operating physicians a method for treating systemic organ tumors by implanting the multi-delivery catheter adjacent to a tumoral organ. This allows locus specific delivery of gene therapy, cell therapy, and other therapeutic agents without direct implantation of the multi-delivery catheter into an organ. This mode of therapy is especially useful in systemic cancers not capable of an adequate surgical resection either because of the risk of the procedure to the patient, or the inability to obtain an adequate surgical margin.

Finally, it is yet another object of the method to provide preoperative simulation of the infusion of gene therapy agent vectors and other intratumoral infusates to maximize infusion efficiency and minimize local toxicity to the adjacent cellular tissue. The diffusion model permits a systematic design of targeted delivery into the tumor by predicting achievable volumes of distribution for therapeutic agents based on the established transport and chemical kinetics models. The model can be simulated in a computer-aided brain analysis before the actual placement procedure, thus reducing the need for trial-and-error animal experimentation or intuitive dosing in human trial. Computer-aided simulation will maximize preoperative planning, and minimize intraoperative and postoperative complications. Further, a convection enhanced delivery (CED) model will also be available to the clinician. Through CED, the clinician can design a treatment that distributes therapeutic agents much farther into organ or tissue than diffusion. With the convenience of the disclosed invention, CED can now be applied in cycles of gene therapy delivery with prodrug treatment in viruses containing cytotoxic genes. This allows the clinician to apply real-time customizable treatment options based upon closed-loop biofeedback parameters.

While the apparatus and method has or will be described with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention used in this method can be better visualized by turning now to the following drawings wherein like elements are referenced by like numerals.

In summary, according to this invention, this method utilizes a fully internalized, surgically implanted pump and multi-delivery catheter device whereby: viral or non-viral gene therapy, or cell therapy, is delivered locally to a tumor or tumor resection site; is usually in conjunction various adjunctive treatments also administered locally; where the delivery is controlled externally; and various feedback testing on the pumping device and external to it are used in order to provide patient specific treatment regimes.

### BRIEF DESCRIPTION OF THE DRAWINGS

FIG. 1a is a diagrammatic cross sectional view of a patient's body after the catheter and pump unit have been successfully implanted beneath the skin in the chest cavity or alternatively in the abdominal area of the patient.

FIG. 1b is a block diagram of the architecture of the external controller unit which communicates with the it planted apparatus.

FIG. 1c is a schematic diagram which illustrates the implantable pump and its associated communications controller.

FIG. 1d is a left-lateral cross sectional view of the patient's skull and brain, showing the tumor site location and the implantable catheter.

FIG. 2 is a partial cut away view of the pumping device.

FIG. 3a is a front view of the pumping device.

FIG. 3b is a top view of the pumping device.

FIG. 3c is a back view of the pumping device.

FIG. 3d is a left side view of the pumping device.

FIG. 4a is a back view of the pumping device highlighting the delivery connector.

FIG. 4b is a magnified view of the delivery connector of FIG. 4a.

FIG. 4c is a side view of the pumping device with the septa bump locations highlighted.

FIG. 4d is a magnified cross sectional view of septa locations shown in FIG. 4c.

FIG. 5 is an exploded view of the pumping device.

FIG. 6 is a perspective view of the top of the induction charger assembly and pump electronics assembly coupled together.

FIG. 7a is a perspective view of the top of the pump electronics assembly.

FIG. 7b is a perspective view of the bottom of the pump electronics assembly.

FIG. 8a is a perspective view of the top of the induction charger assembly.

FIG. 8b is a perspective view of the bottom of the induction charger assembly.

The invention used in this method and its various embodiments can now be better understood by turning to the following detailed description of the preferred embodiments which are presented as illustrated examples of the invention defined in the claims. It is expressly understood that the method as defined by the claims may be broader than the illustrated embodiments described below.

### DEFINITIONS

Unless defined otherwise, all technical and scientific terms used herein have the same meanings as commonly understood by one of ordinary skill in the art to which this method belongs. Although any methods and materials similar or equivalent to those described herein can be used in the practice or testing of the present method, the methods, devices, and materials are now described. All publications mentioned herein are incorporated by reference for the purpose of describing and disclosing the materials and methodologies which are reported in the publications which might be used in connection with the invention. Nothing herein is to be construed as an admission that the method is not entitled to antedate such disclosure by virtue of prior invention.

The following mathematical symbols used here in refer to its definitions as follow: Q is infusate flow rate; ρ is fluid density; \vec{ν}_{ƒ} is fluid velocity vector in the catheter; μ is fluid viscosity; ε is tissue porosity; P is infusion fluid pressure; \vec{∇}ₚ is pressure gradient; D_{b} is bulk diffusivity; Dₑ is effective diffusion tensor; C_{ƒ} is the concentration of a drug; \vec{ν}ₜ is fluid velocity in the porous tissue; Dₑ is mean effective diffusivity; k is first order rate constant accounting for drug reaction; [symbol] is Hydraulic conductivity tensor, which is a function of fluid viscosity μ and effective tissue permeability tensor κ; \vec{ν}ₜ, \vec{∇}Cₜ is convection term; [symbol] ₑ \vec{∇}Cₜ is diffusion flux; Cₜ(\vec{x},t) is tissue averaged species concentration; R(Cₜ, \vec{x}) is drug decomposition due to metabolic reaction; and is sink term due to bio-elimination.

The term “drug” is defined under 21 U.S.C. 321§201 as: (A) articles recognized in the official United States Pharmacopoeia, official Homoeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals; and (D) articles intended for use as a component of any article specified in clause (A), (B), or (C). Experimental drugs such as gene therapy and cellular therapy treatments are explicitly included in the term “drug” as utilized in this document.

Gene therapy involves the insertion of genetic material into an individual patient's tissues and cells to treat disease. The medium of transmission of the genetic material into the cell is called a vector. Vectors are often used to transport these genes.

Cell therapy involves the process of introducing whole cells into a tissue in order to provide treatment for a malady or disease. A commonly known example of this would be use of stem cells. Cell therapy is often combined with gene therapy techniques to increase the biocompatibility of the cells introduced to the patient.

Vectors are plasmids, viruses, or bacteria used to contain a certain gene, transport it into host cells and in some cases, facilitate the integration of the gene into the host cell's genome.

A retrovirus is a type of virus that, when not infecting a cell, stores its genetic information on a single-stranded ribonucleic acid (RNA) molecule instead of the more usual double-stranded deoxyribonucleic acid (DNA) molecule. After a retrovirus penetrates a cell, it constructs a DNA version of its genes using a special enzyme called reverse transcriptase. This DNA then becomes part of the cell's genetic material.

An adenovirus is a double-stranded DNA virus commonly used as a vector in gene therapy.

Electroporation is a method of passing a small electric current across the membrane of a cell in order to induce DNA uptake through the temporary and reversible formation of surface pores.

Matrix metalloproteinases are zinc-dependent endopeptidases and are capable of degrading all kinds of extracellular matrix proteins. The can also can process a number of bioactive molecules. They are known to be involved in the cleavage of cell surface receptors and are also thought to play a major role on cell behaviors such as cell proliferation, migration (adhesion/dispersion), differentiation, angiogenesis, apoptosis and host defense. In the therapeutic setting, metalloproteinases are utilized to increase the ease of transfer across a cellular membrane.

### DETAILED DESCRIPTION OF THE PREFERRED EMBODIMENTS

The apparatus utilized in the current method is additionally described and disclosed within U.S. Pat. No. 7,799,012, titled ‘A Magnetic Breather Pump and a Method for Treating a Brain Tumor Using the Same’, issued Sep. 21, 2010, which is herein incorporated by reference in its entirety.

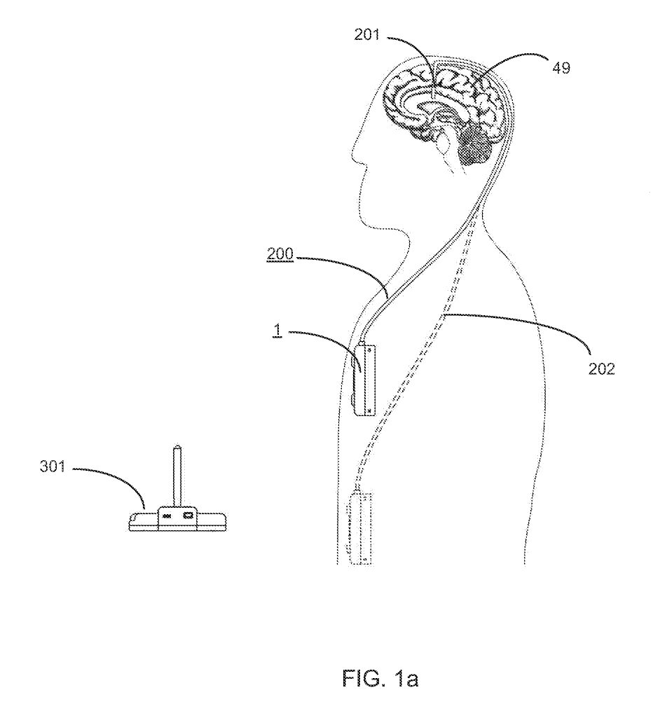

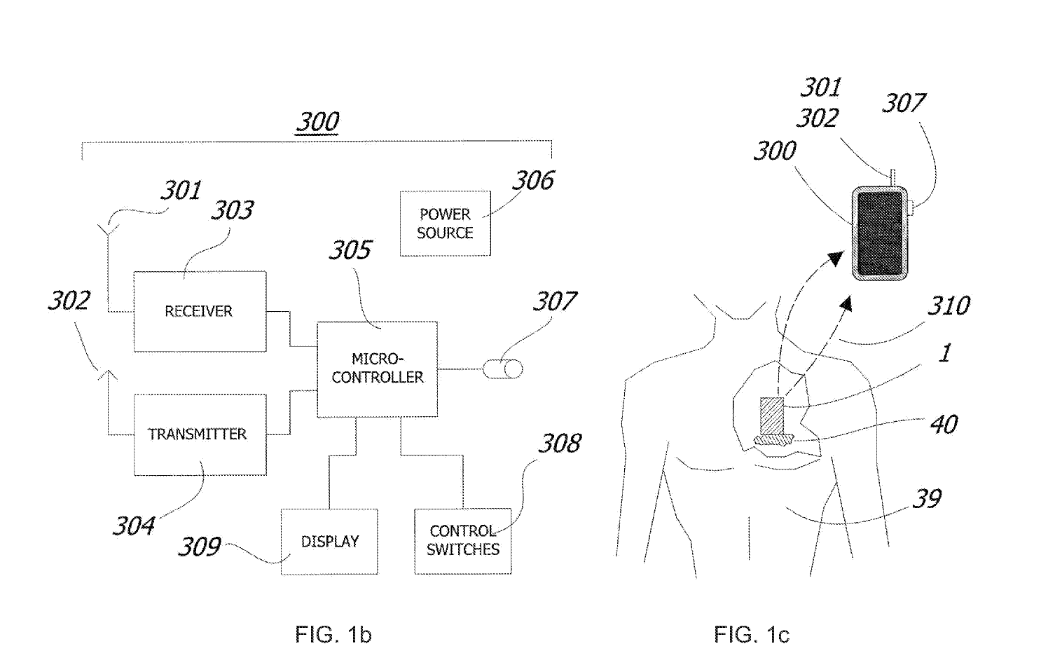

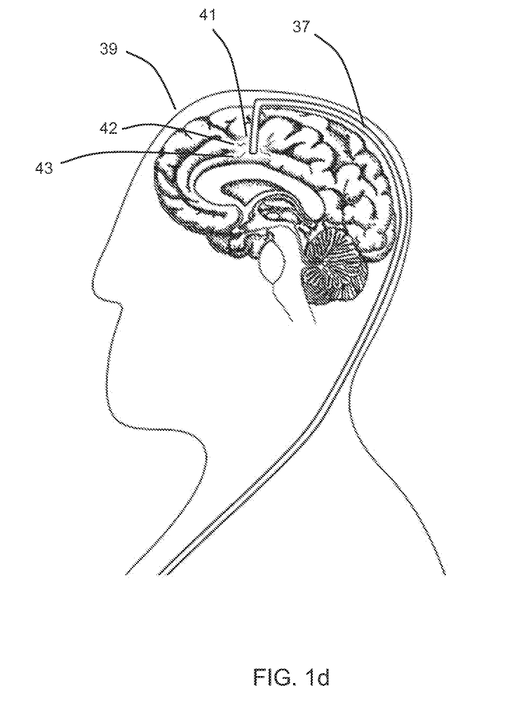

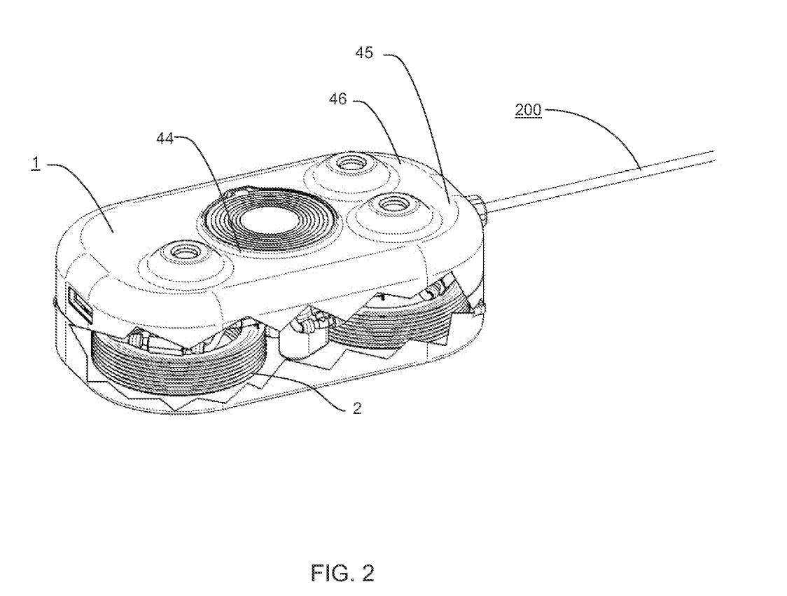

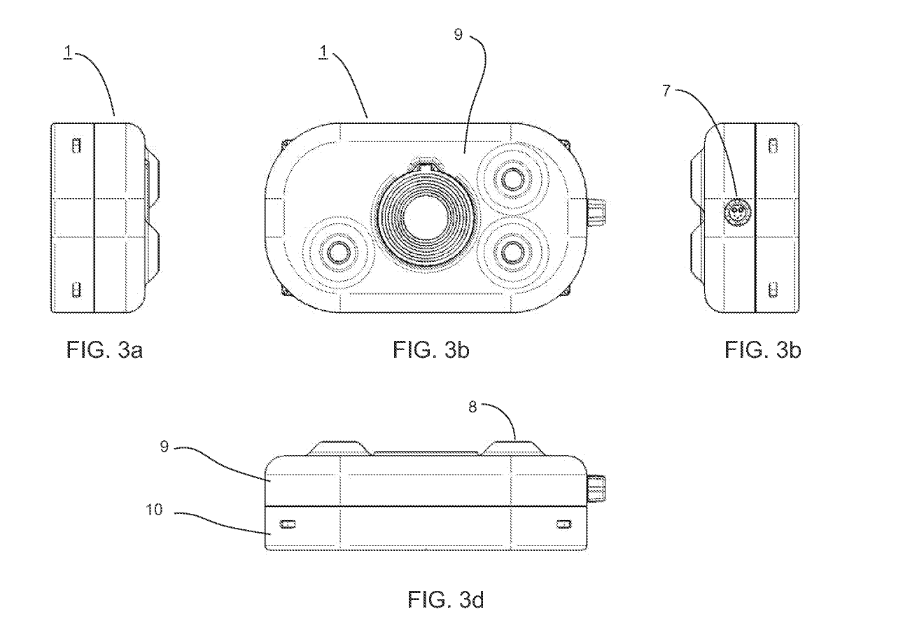

A pumping device 1 as seen in FIGS. 1a and 2 comprises a plurality of multiple pressure-regulated bellows 2 filled with a therapeutic agent to deliver sequential, programmable treatment regimes designed specifically for that individual patient. If the patient's therapy regime necessitates administration of a plurality of therapeutic fluid varieties, then the bellows 2 can be used in sequence and then replaced with new therapeutic substances. The bellows 2 may be refilled transdermally utilizing a syringe and needle. This allows the pumping device 1 to be a vehicle for an infinitely complex combined modality treatment. For example, utilizing this method allows a pumping device 1 comprising four bellows 2 to be used to distribute an unlimited amount different medications and therapies, but with a maximum of four at any one time. The pump device 1 as seen in FIG. 2 shows two bellows 2 in addition to a waste reservoir utilizing a flexible membrane (not seen). However, the inclusion of additional bellows 2 either internally to the pumping device 1 housing or externally tethered via a catheter to the pumping device 1 may be present without departing from the original spirit and scope of the invention.

The current method also incorporates a flushing procedure, by which the plumbing of the pumping device 1 is cleansed internally while still remaining implanted in the patient. This is accomplished by placing saline solution or another biocompatible cleansing agent in one or more of the bellows 2, then pumping the solution through and out of the pump 1. Flushing helps limit the build-up of residues within the pumping device 1 if performed regularly. The current method could also be utilized to eliminate blockages within the plumbing of the pumping device 1, should they occur. Furthermore, if a proper solution is used, then the patient is given the further therapeutic effect of tumoral irrigation. The pumping device 1 would allow locus specific low doses of gene therapy vector or cell therapy solution, leading to more effective treatment that is less catastrophic for the patient. Specifically, low doses of metronomically delivered therapeutic agent may minimize immune response to the treatment. Also, direct intratumoral injection cancels systemic elimination of the gene therapy and cell therapy vector via hepatic metabolism.

The pumping device 1 would be an effective tool in research and development of new gene therapy and cell therapy techniques for cancer treatment. Such direct metronomic intratumoral implementation would only be possible with the use of the above disclosed pump 1.

The pumping device 1 is capable of delivering therapeutic agents in a fully programmable manner specific to the needs of the patient. This may necessitate therapeutic agent to be delivered in a rapid manner over a short period or in a slow yet constant delivery over a long period. This allows the clinician to determine the delivery of therapeutic agents that would be most effective. For instance, for the initial delivery of a gene therapy vector, the clinician may choose to administer a liberal dose of vector in order to reach a critical vector/viral load in the area of the tumor. This might be followed by a one-week treatment of low-dose but constant administration of immunosuppressive/chemotherapy therapy, followed by another large dose of gene therapy vector. By providing the ability to tailor the modality regime to the patient and deliver therapeutic agents directly to the tumor mass, treatment options will be greatly increased.

Turning to FIG. 1a, a delivery hose 200 is coupled to a seal connector and the pumping device 1. The delivery hose 200 thus serves as a conduit between a tumor site 41, houses a return sample fluid line, and contains several electronics connections for various sensors.

Conditions such as cancer may be treated utilizing the implantable pumping device 1. After the cranium of the patient has been opened and the skull and dura have been successfully breeched, the tumor, or as much of the tumor as possible, is removed. The delivery hose 200 is then passed into the tumor site 41 or area of treatment beneath both the dura and skull. The delivery hose 200 then leads away from the distal tip and down the back of the neck of the patient underneath the skin as best seen in FIG. 1d. The delivery hose 200 lies beneath the scalp of the patient for the entire distance between the seal connector on the pumping device 1 and the tumor site 41. The purpose for maintaining the delivery hose 200 beneath the scalp is to give the patient a sense of normalcy and confidence while they are undergoing treatment. It also maximizes normal function of the patient. Furthermore, the risk of infection is reduced because the pumping device 1 and delivery hose 200 are fully implantable, so that the epidermis of the patient is not semi-permanently breeched. In treatments that are not fully implantable, such as standard central IV lines and externalized intracerebral catheters, great care must be taken to sanitize and protect the location where the treatment enters into the body.

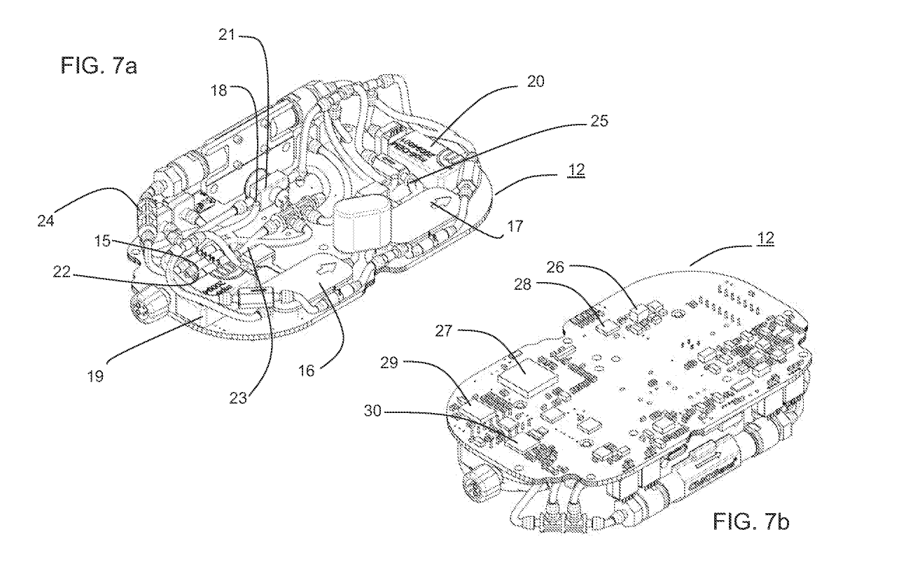

FIG. 1c shows an external controller 300 which communicates with the pumping device 1, FIG. 1b is a block diagram of the external controller 300 and its various components. The pumping device 1 communicates with the external controller 300 by the use of an RF transmitter 304 its associated antenna 302, and an RF receiver 303 with its associated antenna 301. Once the pumping device 1 is implanted subcutaneously in the patient 39 and is in operation, the clinician may decide to change the parameters of the operation. For example, the clinician may change the amount of medication dispensed onto the tumor site 41 or the time intervals associated with the dispensing process. The clinician communicates with the internal electronics of pumping device 1 using the external controller 300 shown in FIG. 1b. The external controller 300 may be in the form of a desktop computer, a personal computing device such as a smartphone, or any other similar appropriate device known in the art. The external controller 300 can also function as a data collection and analysis unit. The external controller 300 is able to communicate with the pumping device 1 through its own microcontroller 305 via RF transmitter 304. RF transmission is accomplished by use of a RF antenna 302, and the RF receiver 303 and its antenna 301. Communication may also be accomplished using the serial communication port 307 which is located in the external controller 300. These new command data sets are then stored in the memory of a microcontroller 27 within the pumping device 1 as seen in FIG. 7b, which is now programmed anew to perform the newly encoded procedural instruction set.

In one embodiment, the external controller 300 is used as a method of implementing preoperative simulation computer software regarding the infusion of gene therapy, cell therapy, and other intratumoral infusates. Preoperative computer simulation diffusion modeling will assist the clinician in maximizing infusion efficiency and minimizing local toxicity to the adjacent tissue from leakage of the infusate into the normal tissue. The term ‘diffusion model’ is meant within this document to describe fluidic therapeutic agent dispersion within tissues utilizing diffusion and/or convection. The term ‘diffusion model’ within this document specifically includes use of convection enhanced delivery (CEO) methods. Specific factors considered in this model are brain geometry, drug and vector properties, catheter dimensions and placement, injection method, drug decomposition, chemical kinetic reaction, and bio-elimination. Other variables can be incorporated to improve the accuracy of the prediction model based upon the specific treatments and therapeutic agents utilized.

In the first step of malignant glioma modeling, the patient-specific diffusion tensor imaging (a method of MRI) is used to construct a brain tumor model with accurate geometry (sharp boundaries and surfaces of the substructures). In the second step, the brain region is partitioned into small discrete volume grids. In the third step, a set of equations and boundary conditions describe flow physics and mass transfer between the finite volumes in the brain region. In the final step, the equations are solved numerically over the finite volume and the boundaries between the adjacent volumes. This same process could also be utilized for tumor bodies not located in the cranium.

The patient specific imaging data not only provides the accurate size and shape of the tumor region, but also permits reconstruction of physiologically consistent substructures and boundaries between regions in the brain or organ. Tissue properties (such as porosity, tortuosity, diffusivity, permeability, and hydraulic conductivity) can be estimated from the brain location and reference literature. These parameters, combined with the delivery hose 2000 placement and orientation with respect to the tumor region, allow estimation of location-specific parameters. These location specific parameters include such factors as diffusivity tensor, permeability tensor, and hydraulic conductivity tensor values. Once critical patient-specific factors are known, they can be entered into the flow and mass transfer equations specific to those factors. With this information, an accurate model of patient treatment can be produced. The clinician is then empowered to model various treatment regimes in order to find the most efficacious therapy modality.

The brain, including the tumor region, is partitioned into small triangular and quadrilateral elements using Delaunay triangulation. Each small finite volume is linked to its neighbors so as to form a logically connected computational mesh, which can be generated by grid generation software such as Fluent software (by ANSYS, Inc.) or other Computational Fluid Dynamics (CFD technology) methods. The grid sizes need to be large enough to minimize the number of volume elements for calculations yet small enough to be able to spatially resolve the anatomical properties of the tumor area. A typical simulation consists of approximately 20,000 to 30,000 volume elements distributed in the region covering about one quarter of the brain (300 cc). The flow and mass transfer equations are enforced over the computational domain consisting of these meshes.

The therapeutic agent's delivery to the brain is simply modeled as inserting solution consisting of the vectors or drug solutes into porous brain tissues via an infusion catheter. The solution is assumed to be an incompressible Newtonian fluid. Motion can be described by the mass and momentum conservation equation. Additionally, the drug distribution is described by the species transport and chemical kinetics equations. The diffusion model consists of two parts; the flow inside the catheter, and the flow in porous brain tissues.

For the flow inside the catheter, the model divides the space inside the lumen of the catheter into small finite elements. The fluid flow between the finite elements is modeled with the continuity and Navier-Stokes equations as shown in Equations 1 and 2, respectively. The continuity equation (Eq 1) describes that the fluid is incompressible.

\vec{∇}·(ρ\vec{ν}_{ƒ})=0 (1)

The Navier-Stokes equation (Eq 2) describes that the momentum of the fluid flow is conserved. It states that any change in fluid velocity in the catheter (the left-hand side of the equation) is due to the pressure gradient (caused by the pumps) and resistance of the flow due to fluid viscosity.

$$ρ ( \frac{\partial {\vec{v}}_{f}}{\partial t} + {\vec{v}}_{f} \cdot \vec{∇} {\vec{v}}_{f} ) = - \vec{∇} p + μ {\vec{∇}}^{2} {\vec{v}}_{f} \qquad ( 2 )$$

The movement of the viral vectors, gene therapy solution, cell therapy solution, and drug molecules inside the catheter due to the flow can be modeled with the species transport equation, as shown in Equation 3. It states that the change in concentration of the molecules due to diffusion and convection (the left-hand side of the equation) depends on the divergent of the product of the diffusivity and concentration gradient of the molecules in the fluid.

$$\frac{\partial {C}_{f}}{\partial t} + {\vec{v}}_{f} \cdot \vec{∇} {C}_{f} = \vec{∇} \cdot ( {D}_{b} \vec{∇} {C}_{f} ) \qquad ( 3 )$$

The flow inside the brain is modeled as the fluid flow in a porous medium. The brain is partitioned into small finite elements and the flow between these elements is modeled with the continuity equation and Darcy's Law, as respectively shown in Equations 4 and 5. The continuity equation (Eq 4) describes that the loss of fluid in the flow is due to the absorption into the porous medium. The fluid velocity in tissue is related to average fluid velocity through porous tissue, \vec{ν}ₜ=ε\vec{ν}ₚ, and is dependent on the specific porosity of the tissue. At the tip of the catheter, the average fluid velocity is the same as the fluid velocity corning out of the catheter: \vec{ν}ₚ=\vec{ν}_{ƒ}. The amount of fluid loss captured in the sink term is a function of the difference between the interstitial fluid pressure and the venous pressure: S_{B}=ƒ(p−pᵥ).

\vec{∇}·(ρ\vec{∥}ₜ)=S_{B} (4)

The fluid dynamics in the porous brain is embodied in the Darcy's Law (Eq 5), which states that the momentum of the fluid flow is conserved. It states that any change in fluid velocity in the brain (the left-hand side of the equation) is due to the pressure gradient (caused by the flow out of the catheter) and resistance of the medium to the flow.

$$\frac{ρ}{ɛ} ( \frac{\partial {\vec{v}}_{t}}{\partial t} + {ɛ}^{- 1} ( {\vec{v}}_{t} \cdot \vec{∇} ) {\vec{v}}_{t} ) = - \vec{∇} p - {ℜ}^{- 1} {\vec{v}}_{t} \qquad ( 5 )$$

The movement of the drug molecules inside the brain due to the flow described in Equation 5 can be modeled with the species transport equation as shown in Equation 6. It states that the change in concentration of the molecules due to diffusion and convection (the left-hand side of the equation) depends on the divergent (DIV) of the product of the diffusivity tensor of the brain medium, and concentration gradient of the molecules in the fluid. The accuracy of the model can be improved by incorporating the loss of drug molecules due to decomposition and bio-elimination.

$$ɛ \frac{\partial {C}_{t}}{\partial t} + {\vec{v}}_{t} \cdot \vec{∇} {C}_{t} = \vec{∇} \cdot ( {𝒟}_{e} \vec{∇} {C}_{t} ) + R ( {C}_{t} , \vec{x} ) + S ( {C}_{t} , \vec{x} ) \qquad ( 6 )$$

The completeness of the diffusion model is captured in the boundary condition assumptions listed below. At the catheter inlet, the infusion flow rate or pressure and concentration of drug are assumed to be constant. At the interior wall inside the lumen of the catheter, the flow is assumed no slip,

$$\frac{\partial p}{\partial n} = 0 ,$$

and the drug doesn't penetrate (zero flux) into the catheter wall, \vec{n}, \vec{∇}C_{ƒ}=0 and \vec{ν}_{ƒ}=0. At the outer surface of the catheter, the same boundary conditions are assumed as in the inside. At the catheter tip, the continuity of flow is assumed: \vec{ν}_{ƒ}|ₗᵤₘₑₙ=\vec{ν}_{Cont}=\vec{ν}ₜ, and, pₗᵤₘₑₙ=p_{Cont}, and C_{ƒ}|ₗᵤₘₑₙ=Cₜ. At the lateral ventricles or capillary surfaces, the fluid pressure is the same as the pressure of the cerebrospinal fluid (CSF) or other surrounding fluid. No fluid flow through the ventricle and capillary walls, \vec{n}, \vec{∇}νₛ=0, \vec{n}, \vec{∇}ν_{y}=0. Only the mass transfer through the permeable ventricle and capillary walls is assumed: −Dₑ(\vec{n}, \vec{∇}Cₜ)=k(Cₜ−C_{∞}). Molecule transfer through permeable boundary is only one way; drug molecules can leave but cannot return. Bio-elimination “sink term” is assumed as a function of the difference between interstitial pressure and venous pressure: S_{B}=ƒ(p−pᵥ).

The six partial differential equations (Eq 1-6) are applied to the discrete volumes in the model to produce a set of non-linear algebraic equations for the entire brain model. These equations are solved with proper boundary condition using the iterative Newton-Krylov method and simulated using commercial computational fluid dynamics (CFD) software such as Fluent.

The microcontroller 27 located in pumping device 1 and implanted inside the patient's body 39 communicates with the external controller 300 via RF transmitter 304 and RF receiver 303. This process sends collected data from the pumping device 1 to the external controller 300. This feature enables the clinician to collect data and to determine the state of the patient throughout the period of treatment. These data are stored inside the external controller 300, providing chart history of the treatment status of the parameters associated with the tumor site 41. The pumping device 1 transmits data for collection and storage. The external controller 300 is controlled by the user via the settings in control 308 seen in FIG. 1b. The external controller 300 also displays the amount of vector dispensed over time by the multi-lumen delivery hose 200 on its display 309. Data collected in this manner can be used to correlate the behavior pattern of a particular patient and his or her chart history. A data collection and analysis program can be displayed by the external controller 300. Once the data is collected from the pumping device 1, the external controller 300 or the host PC can then plot the data on a time scale and analyze the data further. Data in the form of the historical plot of cause-and-effect provide significant immediate benefit to the patient 39 and aide in future research. The entire external controller 300 as shown in FIG. 1b is run by power obtained from a power source 306.

FIG. 1c is an illustration of a patient 39 with the implanted pumping device 1. The external controller 300 with its associated serial port 307 and receiver antennae 301 and transmitter antennae 302 is shown in its bidirectional communication mode with the implanted pumping device 1. A suture location 40 is visible where the implanted pumping device 1 would be surgically attached to the patient 39. The external controller 300 and the implanted pumping device 1 communicate via the RF path 310.

FIG. 1d is an illustration of the patient 39 with a multi-lumen catheter 37 implanted in solid brain tissue, the proximal end of the multi-lumen catheter 37 coupled to the delivery hose 200 seen in FIG. 1a. The tumor site 41 is visible, showing the area of disease that requires treatment with gene and cell therapy. The dark flexuous lines represent therapeutic agent delivery to the tumor site 41. Specifically, utilizing gene therapy 42 and cell therapy 43 solutions.

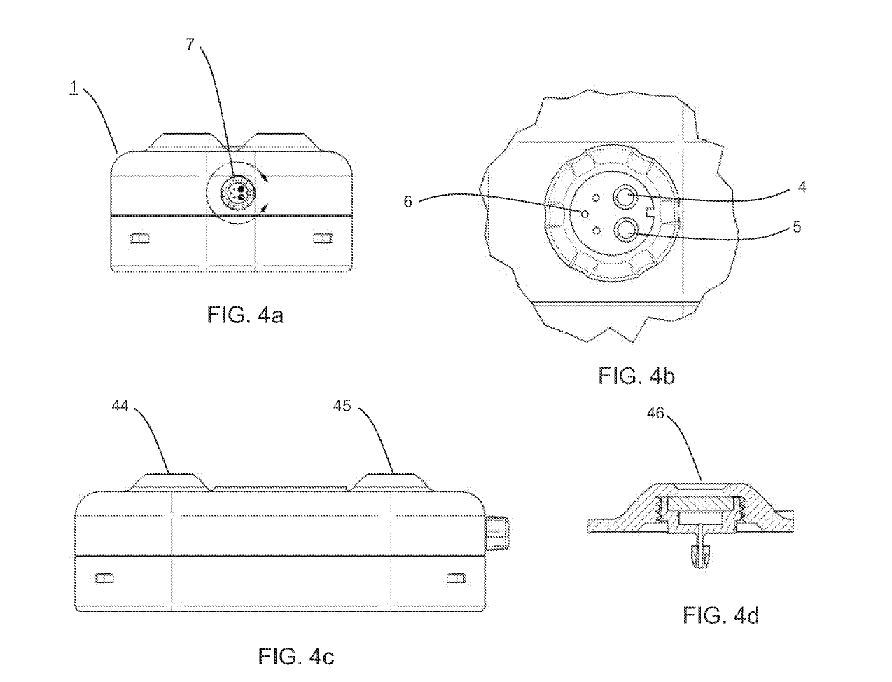

Turning to FIG. 4a, the pumping device 1 comprises a delivery connector 7 where the delivery hose 200 couples with the pumping device 1. The delivery connector 7 contains a drug outlet 4, a sample return 5, and a plurality of sensor connections 6 as seen in FIG. 4b for controlling the pump unit 1 and for analyzing the sample fluid that is obtained from the patient. The drug outlet 4 is the aperture in which gene therapy vector, cell therapy solution, or mixtures of medicating agents with vector, are sent from the pumping device 1 through the delivery hose 200. Similarly, the sample return 5 is the aperture where fluid that has been collected from the patient is returned by the delivery hose 200 and enters the pumping device 1 for analysis. The process by which the pumping device 1 sends the therapeutic agent or agents and receives sample fluid obtained from the patient through the delivery hose 200 is explained in further detail below.

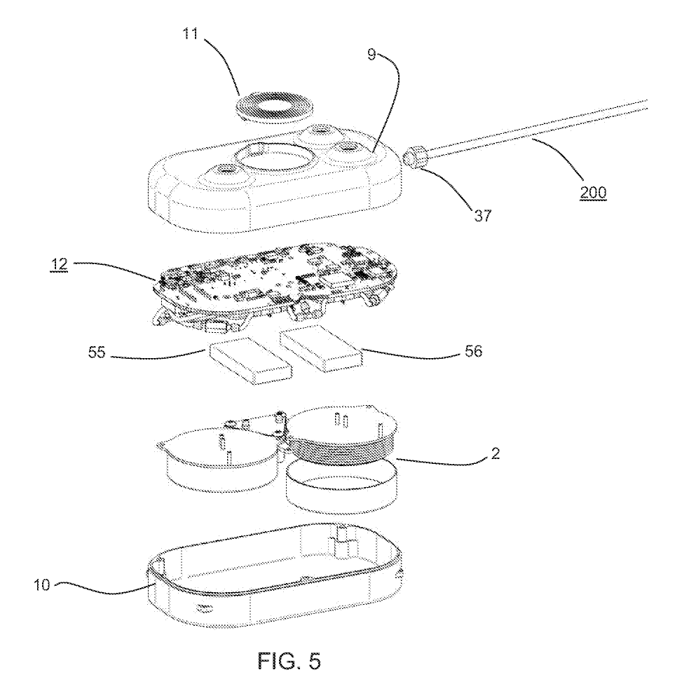

A pair of bellows 2 are housed in the bottom portion 10 of the pumping device 1, which are depicted in FIG. 5. It is to be expressly understood that fewer or additional bellows 2 may be present without departing from the original spirit and scope of the invention. To introduce gene therapy vectors into the pumping device 1, a bellows 2 is filled via its respective septa port 44, 45 (seen in FIG. 4c) with a needle and syringe. A septa fluid flow pathway 18 extends from the interior of the pumping device 1 (shown in FIG. 7a) and forms part of the internal plumbing structure that carries therapy solutions to the delivery site via the delivery hose 200. The pumping device 1 then delivers in the therapeutic solution in a series of steps that are described below.

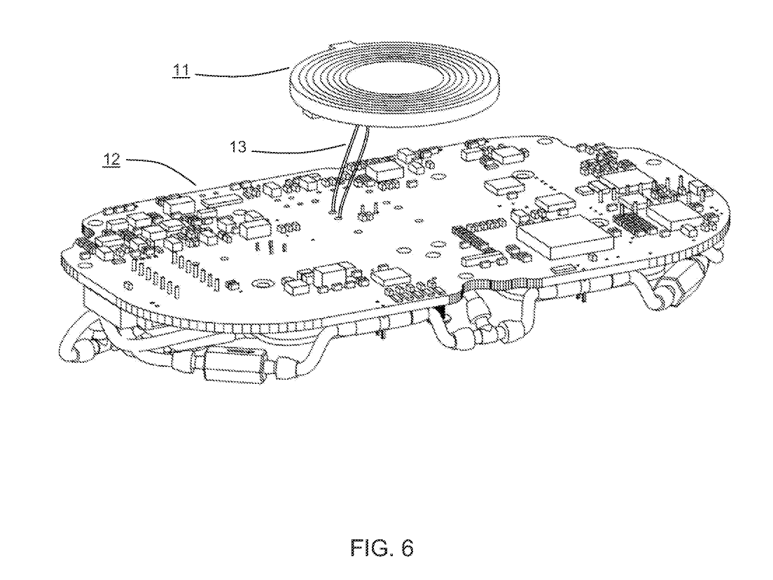

Turning to FIG. 6, the interior of the pumping device 1 is comprised of two assemblies, a pump electronics assembly 12 and an induction charger assembly 11. The pump electronics assembly 12 and the induction charger assembly 11 are both housed within the pumping device 1, and are joined by an electronic interconnect cable 13.

The pump electronics assembly 12 is shown in greater detail in FIGS. 7a and 7b. As seen in FIG. 7b, the pump electronics assembly 12 contains a drug delivery CPU or microcontroller 27 that stores its program and is coupled to data FLASH memory modules 28. The power regulation unit 26 acts as a buffer and controls power to the CPU 27 and other components on the assembly 12. Pre-stored information such as look-up tables and the like are stored on the FLASH memories 28. The CPU 27 runs a pre-installed intelligent delivery software program and controls an ampule pump driver 20, a return pump integrated circuit 19, and a delivery valve drift integrated circuit 22 as seen in FIG. 7a. The drug delivery CPU 27 also communicates with a lab-on-a-chip (LOC) 21 and receives important treatment data. The lab-on-a-chip 21 pictured in FIG. 7a is in the form of a miniature spectrophotometer, and the glass flow cell LED, and light sensor which make up the lab-on-a-chip 21 are visible. Additional lab-on-a-chip technology is expressly envisioned, including but not limited to aptamer, antibody, and half-antibody based biosensor technology.

The drug delivery CPU 27 is pre-programmed and is capable of transmitting data through RF antennae. The RF transceiver 29 is connected to a RF antenna 30. A user or qualified physician who wishes to change the patient's drug regimen from a remote location first sends the data to the patient. The sent information is then picked up by the RF transceiver 29 and antenna 30 and is then stored on the FLASH memory chips 28. When the delivery CPU 27 retrieves information from the FLASH memory chips 28 it adjusts the treatment regimen according to the user's data instructions. Some examples of the treatment regime adjustments that would be applied include changes to dose, scheduling, and therapeutic agent used.

The pumping device 1 is capable of delivering up multiple different drugs simultaneously with high accuracy. The pump electronics assembly 12 (FIG. 7a) comprises up to four piezoelectric pumps 17. These pumps 17 are driven by a corresponding piezoelectric pump driver 20. The pump 17 and corresponding driver 20 work together to pump the therapeutic agent out of the bellows 2. The use and manufacture of piezo pumps is well known to those in the art. Fewer or additional piezo pumps 17 than what is depicted in FIG. 7a may be used without departing from the original spirit and scope of the invention. The piezoelectric pump 17 moves the therapeutic agent through a manifold tube 24, into a delivery valve 15, then out through the drug delivery connector 7. The delivery valve 15 (FIG. 7a) is regulated by a delivery valve driver integrated circuit, which is controlled by the drug delivery CPU 27. The therapeutic agent is pumped through the delivery connector 7 (FIG. 4a) and then enters into the delivery hose connector 37 (FIG. 5) via the drug output 4 (FIG. 4b) located in the delivery connector 7. The therapeutic agent is then pumped through the delivery hose 200 to the treatment site 41.

The pumping device 1 is fully programmable and runs intelligent software to determine what and how much drug is required. The regulation loop of the intelligent drug delivery system uses a return sample of fluids from the “delivery area” to determine the necessary response.

The return sample fluid obtained from the patient travels through the return lumen within the delivery hose 200 (FIG. 5), through the delivery valve 15 (FIG. 7a), through the delivery hose connector 37 (FIG. 5) via the sample return port 5 (FIG. 4b), and then enters the delivery connector 7 (FIG. 4a). After the sample fluid passes from the delivery connector 7 it then moves through the return valve 22 (FIG. 7a). The negative pressure necessary to pump the sample is created by the return piezoelectric pump 16 (FIG. 7a). The pump 16 is powered by a return pump driver 19. The fluid sample then travels from the return valve 22 into a return pump input tube 25, and into the lab-on-a-chip 21. The lab-on-a-chip 21 senses the chemical composition of the sample. The return piezoelectric pump 16 continues pumping the sample fluid to the waste reservoir located between the bellows 2 of the pumping device 1, where the fluid is collected via syringe and needle by the doctor or practitioner assisting the patient. Collected fluid may be subject to further lab testing as needed.

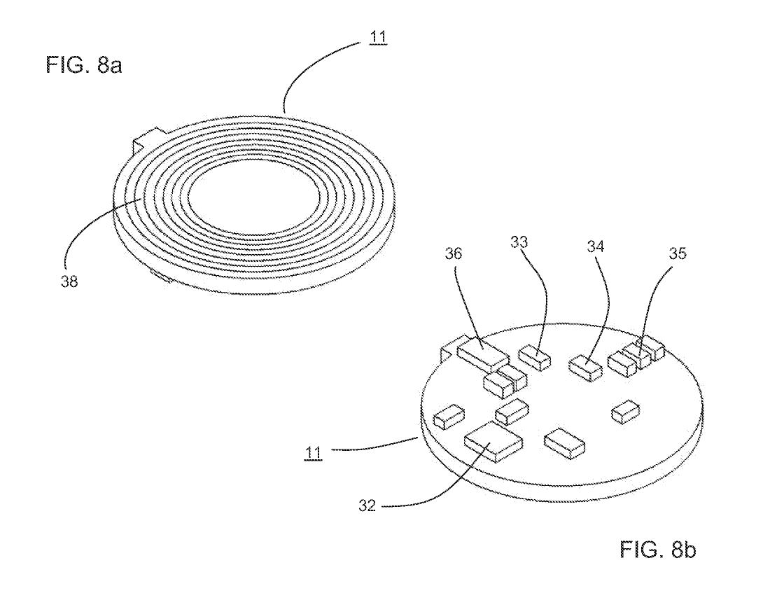

The second main assembly, the induction charger assembly 11, is depicted in greater detail in FIGS. 8a and 8b. The induction charger assembly 11 provides a means for charging a lithium ion battery 55, 56 (shown in FIG. 5). An induction coil 38 coupled to the induction charger electronics assembly 11 receives a high frequency (50 Khz) induced magnetic field from similar charging coil from an external battery charger device (not shown). The induction coil 38 is coupled to a rectifier 35 shown in FIG. 8b. The rectifier 35 converts the high frequency voltage to a DC voltage that is filtered by an inductor 34 and capacitors 33. A battery charger controller 32 regulates the charging of the battery 55, 56 (FIG. 5). A charger connector 36 is utilized for both powering the electronics and charging the lithium ion battery 55, 56. The battery 55, 56 is appropriately sized to provide sufficient power for days of service without the need of charging. Multiple batteries may also be utilized instead of a single battery as a consideration to space and engineering of the device as needed without departing from the original spirit and scope of the invention.

The blood brain barrier, mesothelium, and extracellular matrices are significant as potential natural obstacles to therapeutic agent delivery within the body. These natural obstacles to therapeutic agent delivery are circumvented by use of the pumping device 1 and the combination of the delivery hose 200 and multi-lumen catheter 37 which provide local delivery to brain tissue when needed. The bellows 2 may be filled with a variety of therapeutic agents, allowing for directly intratumoral delivery of combined modality regimes.

Direct intratumoral delivery would allow minimal immune response to locally effective gene therapy and cell therapy treatments. Gene therapy and cell therapy administration can cause an immune response in the patient. This diminishes or destroys the effectiveness of multiple treatments because the patient's immune system will reject the therapy.

Using the disclosed invention allows gene therapy and cell therapy to directly access the malignant tumor cells with diminished systemic immune response. Further, this method bypasses natural barriers such as the blood brain barrier. Delivering the gene therapy vector directly into the tumor also allows for more concentrated doses, which greatly diminishes the side effects associated with the systemic intravenous delivery of the therapeutic agents listed above.

The lab-on-a-chip 21 comprises a means for directly monitoring tumor marker levels and protein levels in the “delivery area.” Tumor markers are specific to the malignancy that is being treated. The lab-on-a-chip 21 is specifically configured for measurements that would be germane to the type of cancer being treated. For instance, VEGF levels are used as measurement of malignant glioma. Growing tumors have high VEGF levels to support vascular growth. As the tumor growth is halted and reversed, lower levels of VEGF will be present in the cerebrospinal fluid. In this example, measuring VEGF level with the lab-on-a-chip 21 allows the clinician to assess the effectiveness of the intratumoral administration of therapeutic agents.

The pumping device 1 is capable of communication with an external controller unit 300 by way of a wireless signal such as RF communication. The 402-405 MHz medical implant communication service (MICS) band could be used to communicate between the controller unit 300 and the pumping device 1. The delivery hose 200 is then operated within the treatment site of the patient in order to infuse the intratumoral therapeutic agent to the treatment site. The delivery hose 200 is then used to suction in a sample of fluid from the treatment site and transfer it to the pumping device 1 and its attendant sensors. The external controller unit 300 is then used to track and monitor the progress of the patient's treatment, and comprises the means for altering and changing the patient's treatment.

Also as similarly described above, the external controller unit 300 is enabled to display the amount of intratumoral therapeutic agent dispensed over time by the delivery hose 200 within the treatment site.

In another embodiment, the method of measuring the effectiveness of the intratumoral therapeutic agent administration further comprises displaying the results obtained from the pumping device 1 unit on a display.

In an alternative embodiment, the external analyzer-pump unit 300 further comprises means for providing a preoperative simulation of the infusion of the intratumoral therapeutic agent, and other intratumoral infusates, to maximize efficiency and minimize toxicity by means of a diffusion model. The diffusion model will also model CED methods.

The external analyzer-pump unit 300 further comprises entering command functions and data into the external analyzer-pump unit 300 from a keyboard and displaying the commands on a display 309. The entering of command functions may comprise sending command functions and data to the external analyzer-pump unit 300 by means of a RF signal transmission, or another form of wireless communication 310.

For treatment of a patient 39, gene therapy 42 and cell therapy 43 would be utilized in a combined modality regime. These treatments could be used in sequence, providing first a way to diminish or abolish the disease (through gene therapy techniques to shrink the tumor and limit metastasis), then repair damage to the organ tissue (example: tumor site 41) where possible (through cell therapy, such as implantation of stem cells).

The use of gene and cell therapy does not preclude the use of other drugs in treatment of the patient, which may be combined with gene and cell therapy for the purpose of maximizing the effectiveness of the treatment to the patient.

Many alterations and modifications may be made by those having ordinary skill in the art without departing from the spirit and scope of the invention. Therefore, it must be understood that the illustrated embodiment has been set forth only for the purposes of example and that it should not be taken as limiting the method as defined by the following method and its various embodiments.

For example, one skilled in the art may produce a device with fewer or additional drug bellows or piezoelectric pumps without departing from the original scope and spirit of the invention.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the method includes other combinations of fewer, more or different elements, which are disclosed in above, even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the method is explicitly contemplated as within the scope of the invention.

The words used in this specification to describe the method and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a subcombination.

Insubstantial changes from the claimed subject matter, as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted, and also what essentially incorporates the essential idea of the method.

What is claimed is:

1. A method for delivering a viral or non-viral gene vector for gene therapy or a therapeutic agent for cell therapy of a tumor in a patient comprising:
surgically implanting a fluid-exchange catheter into a treatment site;
coupling the fluid-exchange catheter to an analyzer-pump unit;
operating the analyzer-pump unit to infuse the viral or nonviral vector or the therapeutic agent stored in a reservoir into the treatment site;
suctioning a sample of fluid from the treatment site;
transferring the sample to the analyzer-pump unit;
monitoring progress of treatment by means of the analyzer-pump unit;
changing the treatment by controlling the analyzer-pump unit; and
refilling or replacing a reservoir containing the viral or nonviral vector or therapeutic agent.

2. The method of claim 1 where operating the analyzer-pump unit comprises contracting and then expanding an inner membrane reservoir in the analyzer-pump unit by oscillation of a magnetic solenoid coupled to the inner membrane reservoir to deliver the gene vector or therapeutic agent.

3. The method of claim 2 where monitoring the progress of treatment by means of the analyzer-pump unit comprises measuring the effectiveness of intratumoral administration.

4. The method of claim 3 where measuring the effectiveness of intratumoral administration further comprises displaying results obtained from the analyzer-pump unit on a display including displaying information related to a determination of effectiveness, an amount of gene vector or therapeutic agent dispensed as a function of time and any flow rate at which the gene vector or therapeutic agent was dispensed.

5. The method of claim 1 further comprising providing a preoperative simulation using a diffusion model or a convection enhanced delivery model of infusion of the viral or nonviral vector or the therapeutic agent, individually or in combination, to maximize efficiency and minimize toxicity.

6. The method of claim where changing the treatment by controlling the analyzer-pump unit further comprises entering a command or data into the analyzer-pump unit from a remote keypad and displaying the commands on a display.

7. The method of claim 1 where changing the treatment by controlling the analyzer-pump unit further comprises sending a command or data to the analyzer-pump unit by means of an RF transceiver and antenna.

8. The method of claim 1 where refilling or replacing the reservoir further comprises refilling or replacing at least four drug ampules included in the analyzer-pump unit, wherein at least one of the four drug ampules is for gene therapy or cell therapy only.

9. The method of claim 1 further comprising cleaning the analyzer-pump unit by refilling the reservoir with an ampule of saline solution or a cleansing agent, pumping the saline solution or cleansing agent through the fluidicly communicated portions of the analyzer-pump unit, thereby preparing the analyzer-pump unit to deliver to the patient a substance that would otherwise be incompatible with substances previously administered by the analyzer-pump unit.

10. The method of claim 9 where contents of the ampules are utilized for combined modality treatments by repetitively cleaning the analyzer-pump unit and refilling or replacing of the reservoir as many times as necessary to utilize a plurality of therapeutic agents including at least one viral or non-viral gene vector for gene therapy or therapeutic agent for cell therapy.

11. The method of claim 1 where the viral vector comprises a retrovirus, which includes a virus from the subclass lentivirus; adenovirus; adeno-associated virus; and man-made virus, including a chimera or hybrid virus including a VSV G-pseudotyped lentivirus.

12. The method of claim 1 where the vector comprises either a replication competent or replication incompetent vector.

13. The method of claim 11 where the vector is utilized in conjunction with a cell insertion technique, including electroporation, sonoporation, or use of a gene gun.

14. The method of claim 1 where the non-viral vector comprises naked DNA, an oligonucleotide, a lipoplex, or a polyplex used in conjunction with an endosome-lytic agent, dendrimer, a hybrid method for creation of a vector including a virosome, a nanoengineered substance including an or ormosil or a bacteria.

15. The method of claim 1 where monitoring progress of the patient's treatment further comprises passing the sample of fluid through a means for fluid analysis in the analyzer.

16. The method of claim 1 where the vector is utilized in conjunction with a cell insertion technique including electroporation, sonoporation, or use of a gene gun.

17. The method of claim 1 where cell therapy includes the use of an allogeneic or autologous stem cell, a mesenchymal stem cell, an animal source for xenotransplantation or the patient's own differentiated cells utilized to create a transdifferentiated cell.

18. The method of claim 1 where the viral vector comprises a retrovirus, including a lentivirus, an adenovirus, an adeno-associated virus; or a man-made virus, including a chimera or hybrid virus including a VSV G-pseudotyped lentivirus.

19. An apparatus for delivering a viral or non-viral gene vector for gene therapy or a therapeutic agent for cell therapy of a tumor in a patient comprising:
a fluid-exchange catheter adapted for surgical implantation into a treatment site;
an analyzer-pump unit fluidicly communicated with the fluid-exchange catheter;
a refillable or replaceable reservoir fluidicly communicated with the analyzer-pump unit to infuse the viral or nonviral vector or the therapeutic agent stored in a reservoir into the treatment site;
where the analyzer-pump unit suctions a sample of fluid from the treatment site, analyzes the sample to monitor the treatment,
where the analyzer-pump unit is controllable to change the treatment in response to monitoring.

20. The apparatus of claim 19 where the analyzer-pump unit comprises an inner membrane reservoir operable by oscillation of a magnetic solenoid coupled to the inner membrane reservoir to deliver the gene vector or therapeutic agent.
